(19) United States
(12) Patent Application Publication
(10) Pub. No.: US 2024/0068946 A1
(43) Pub. Date: Feb. 29, 2024

(54) NANOSTRUCTURED SYSTEM FOR FLUOROMETRIC NUCLEIC ACID AMPLIFICATION

(71) Applicant: Samsung Electronics Co., Ltd., Gyeonggi-do (KR)

(72) Inventors: Vinayak NARASIMHAN, Altadena, CA (US); Radwanul Hasan SIDDIQUE, Monrovia, CA (US); Wonjong JUNG, Seoul (KR); Kak NAMKOONG, Seoul (KR); Youngzoon YOON, Kyonggi-do (KR)

(21) Appl. No.: 17/980,315

(22) Filed: Nov. 3, 2022

Related U.S. Application Data
(60) Provisional application No. 63/402,751, filed on Aug. 31, 2022.

(51) Int. Cl. G01N 21/64; B82Y 5/00; C12Q 1/6844

(52) U.S. Cl. CPC G01N 21/648; B82Y 5/00; C12Q 1/6844; G01N 21/6428

(57) ABSTRACT

An amplification reaction chamber and a method for nucleic acid (NA) amplification in a gene analysis system are provided. The amplification reaction chamber includes an interior surface within which an NA amplification reaction is performed. The amplification reaction chamber also includes a multi-layered nanostructure coating conformally applied to at least a portion of the interior surface and including sub-micrometer nanostructures that enhance fluorescence in the NA amplification reaction based on at least one of plasmonic material of which the sub-micrometer nanostructures are made and geometric dimensions of the sub-micrometer nanostructures.

### PRIORITY

This application is based on and claims priority under 35 U.S.C. § 119(e) to U.S. Provisional Patent Application Ser. No. 63/402,751, filed on Aug. 31, 2022, the contents of which are incorporated herein by reference.

### TECHNICAL AREA

The present disclosure relates generally to nucleic acid (NA) amplification-based diagnostics, and more particularly, to a nanostructured system for fluorometric NA amplification.

### BACKGROUND

Within a gene analysis system, a solution including a bio-sample and lysis buffer is combined with an amplification reagent in an amplification reaction chamber to form an amplification reaction mixture. This combination results in an amplification reaction in which copies of a target NA within the solution are cyclically created.

During NA amplification, the temperature of the chamber is controlled and monitored, and fluorescent labels or reporters (probes and/or dyes) may bind to target sequences in the amplified NA. Excitation light is radiated onto the chamber to allow for radiative coupling with the fluorescent labels, and a resulting optical signal may be detected. Resulting optical signals may include, but are not limited to, fluorescence, color, and turbidity. Accordingly, detection of the optical signal indicates the presence of the target sequence within the amplified NA.

NA amplification strategies include, but are not limited to, polymerase chain reaction (PCR), loop-mediated isothermal amplification (LAMP), nucleic acid sequence based amplification (NASBA), strand displacement amplification (SDA), multiple displacement amplification (MDA), rolling circle amplification (RCA), ligase chain reaction (LCR), helicase dependent amplification (HDA), ramification amplification method (RAM), recombinase polymerase reaction (RPA), and whole genome amplification (WGA).

### SUMMARY

Embodiments provide a nanostructure integration to NA amplification reaction chambers. Specifically, this disclosure provides nanostructures that enhance a fluorescence signal from a reaction mixture in the chamber in order to aid in early conclusive optical detection.

According to an embodiment, a multi-layered nanostructure coating is provided that includes a metal layer, a dielectric layer disposed on the metal layer, and a nanostructure layer disposed on the dielectric layer. The nanostructure layer includes sub-micrometer nanostructures that enhance fluorescence in an NA amplification reaction based on at least one of plasmonic material of which the sub-micrometer nanostructures are made and geometric dimensions of the sub-micrometer nanostructures.

According to an embodiment, an amplification reaction chamber of a gene analysis system is provided. The amplification reaction chamber includes an interior surface within which an NA amplification reaction is performed. The amplification reaction chamber also includes a multi-layered nanostructure coating conformally applied to at least a portion of the interior surface and including sub-micrometer nanostructures that enhance fluorescence in the NA amplification reaction based on at least one of plasmonic material of which the sub-micrometer nanostructures are made and geometric dimensions of the sub-micrometer nanostructures.

According to an embodiment, a method is provided for NA amplification in a gene analysis system. An amplification reaction chamber is conformally coated with a multi-layered nanostructure coating including sub-micrometer nanostructures. An amplification reagent and a solution including at least a bio-sample are combined in the amplification reaction chamber to perform NA amplification. A fluorescence signal from an NA amplification reaction is detected from the amplification reaction chamber. The fluorescence signal is enhanced based on at least one of plasmonic material of which the sub-micrometer nanostructures are made and geometric dimensions of the sub-micrometer nanostructures.

### BRIEF DESCRIPTION OF THE DRAWINGS

The above and other aspects, features, and advantages of certain embodiments of the present disclosure will be more apparent from the following detailed description, taken in conjunction with the accompanying drawings, in which:

FIG. 1 is a diagram illustrating relative fluorescence versus a number of cycles during NA amplification;

FIG. 2 is a diagram illustrating nanostructure integration to a PCR tube or well-plate;

FIG. 3 is a diagram illustrating nanostructure integration to PCR microarrays, according to an embodiment;

FIG. 4 is a diagram illustrating nanostructure integration to a lab-on-a-chip platform, according to an embodiment;

FIGS. 5A and 5B are diagrams illustrating nanostructures for fluorescence enhancement, according to an embodiment;

FIG. 6 is a diagram illustrating a comparison of fluorescence versus a number of cycles during NA amplification with and without nanostructures, according to an embodiment;

FIGS. 7A and 7B are diagrams illustrating an end-point digital assay comparison of fluorescence with and without nanostructures, according to an embodiment;

FIG. 8 is a diagram illustrating film optimization for coating an amplification reaction chamber, according to an embodiment;

FIG. 9 is a diagram illustrating a comparison of fluorescence versus a number of cycles during NA amplification at different fluorescence enhancement, according to an embodiment;

FIG. 10 is a flowchart illustrating a method for NA amplification in a gene analysis system, according to an embodiment; and

FIG. 11 is a block diagram illustrating a controller for controlling a gene analysis system, according to an embodiment.

### DETAILED DESCRIPTION

Hereinafter, embodiments of the present disclosure are described in detail with reference to the accompanying drawings. It should be noted that the same elements will be designated by the same reference numerals although they are shown in different drawings. In the following description, specific details such as detailed configurations and components are merely provided to assist with the overall understanding of the embodiments of the present disclosure. Therefore, it should be apparent to those skilled in the art that various changes and modifications of the embodiments described herein may be made without departing from the scope of the present disclosure. In addition, descriptions of well-known functions and constructions are omitted for clarity and conciseness. The terms described below are terms defined in consideration of the functions in the present disclosure, and may be different according to users, intentions of the users, or customs. Therefore, the definitions of the terms should be determined based on the contents throughout this specification.

The present disclosure may have various modifications and various embodiments, among which embodiments are described below in detail with reference to the accompanying drawings. However, it should be understood that the present disclosure is not limited to the embodiments, but includes all modifications, equivalents, and alternatives within the scope of the present disclosure.

Although the terms including an ordinal number such as first, second, etc. may be used for describing various elements, the structural elements are not restricted by the terms. The terms are only used to distinguish one element from another element. For example, without departing from the scope of the present disclosure, a first structural element may be referred to as a second structural element. Similarly, the second structural element may also be referred to as the first structural element. As used herein, the term “and/or” includes any and all combinations of one or more associated items.

The terms used herein are merely used to describe various embodiments of the present disclosure but are not intended to limit the present disclosure. Singular forms are intended to include plural forms unless the context clearly indicates otherwise. In the present disclosure, it should be understood that the terms “include” or “have” indicate existence of a feature, a number, a step, an operation, a structural element, parts, or a combination thereof, and do not exclude the existence or probability of the addition of one or more other features, numerals, steps, operations, structural elements, parts, or combinations thereof.

Unless defined differently, all terms used herein have the same meanings as those understood by a person skilled in the art to which the present disclosure belongs. Terms such as those defined in a generally used dictionary are to be interpreted to have the same meanings as the contextual meanings in the relevant field of art, and are not to be interpreted to have ideal or excessively formal meanings unless clearly defined in the present disclosure.

The terms used in the present disclosure are not intended to limit the present disclosure but are intended to include various changes, equivalents, or replacements for a corresponding embodiment. With regard to the descriptions of the accompanying drawings, similar reference numerals may be used to refer to similar or related elements. A singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, terms such as “1^{st},” “2nd,” “first,” and “second” may be used to distinguish a corresponding component from another component, but are not intended to limit the components in other aspects (e.g., importance or order). It is intended that if an element (e.g., a first element) is referred to, with or without the term “operatively” or “communicatively”, as “coupled with,” “coupled to,” “connected with,” or “connected to” another element (e.g., a second element), it indicates that the element may be coupled with the other element directly (e.g., wired), wirelessly, or via a third element.

As used herein, the term “module” may include a unit implemented in hardware, software, firmware, or combination thereof, and may interchangeably be used with other terms, for example, “logic,” “logic block,” “part,” and “circuitry.” A module may be a single integral component, or a minimum unit or part thereof, adapted to perform one or more functions. For example, according to one embodiment, a module may be implemented in a form of an application-specific integrated circuit (ASIC), a co-processor, or field programmable gate arrays (FPGAs).

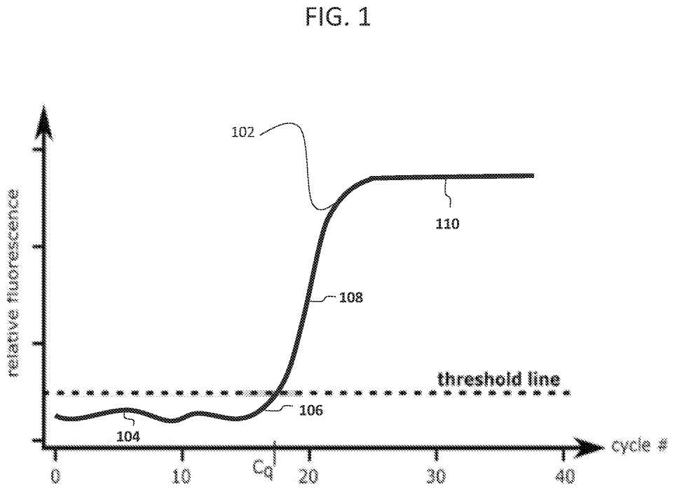

FIG. 1 is a diagram illustrating relative fluorescence versus a number of cycles during NA amplification, according to an embodiment. The presence or absence of fluorescence signals beyond a certain threshold of relative fluorescence is used to determine the presence or absence of a target sequence in the amplified NA. A minimum number of cycles C_{q} of creating copies of a target NA may be required to overcome this detection threshold.

A fluorescence signal 102 begins in a baseline phase 104, where relative fluorescence remains fairly constant over an initial number of cycles. The relative fluorescence then grows in an exponential phase 106, during which the fluorescence signal from the assay overcomes the detection threshold. The fluorescence signal then enters a linear phase 108 in which relative fluorescence grows at a steady rate, and subsequently, a plateau phase in which relative fluorescence peaks and remains constant. In order to obtain a large enough optical signal for conclusive detection, the fluorescence signal generally needs to progress beyond the linear phase. For an NA amplification method (e.g., PCR), this required progression of the fluorescence signal may result in an assay time of approximately one hour. Further, this assay time does not include times required for sample pre-processing and result processing.

NA amplification may be performed in several types of amplification reaction chambers, including, but not limited to, reaction tubes, well plates, microarrays, and a lab-on-a-chip. Nanostructures may be conformally integrated onto all forms of NA amplification reaction chambers in order to enhance a fluorescence signal from the reaction mix and aid in early conclusive detection of the target sequence in the amplified NA. Nanostructures provide a combination of electric-field enhancement and fluorophore quantum-yield enhancement that collectively leads to an overall plasmon-enhanced fluorescence signal.

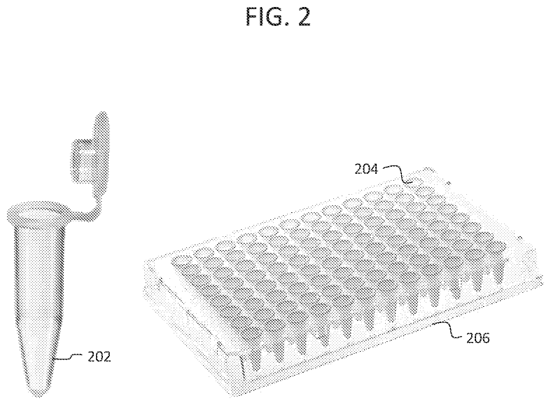

A conformal sub-micrometer multi-layered nanostructure coating may be provided on microfluidic PCR platforms, as shown in FIGS. 2-4. Although coating examples are provided with respect to PCR, embodiments are not limited thereto and similar coatings may be applied in other NA amplification strategies. FIG. 2 is a diagram illustrating nanostructure integration to an individual PCR tube or a well-plate, according to an embodiment. Specifically, the multi-layered nanostructure coating is conformally applied on an interior of an individual PCR tube 202 or each chamber 204 of a well-plate 206. Conformal application refers to a coating that conforms to the contours of the applied surface. The coating may fully or partially cover the interior of the tubes or chambers.

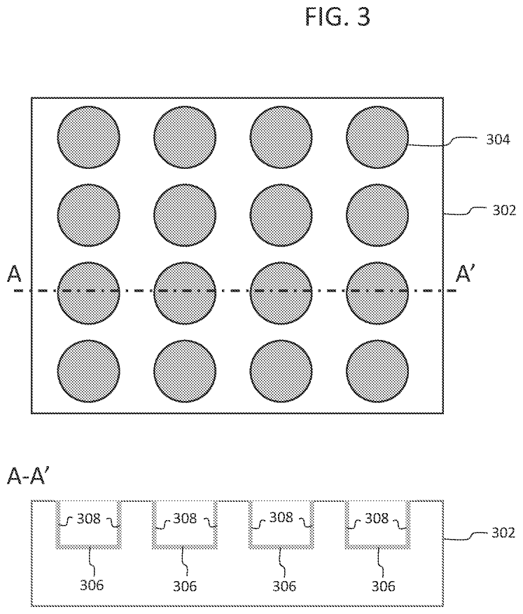

FIG. 3 is a diagram illustrating nanostructure integration to PCR microarrays, according to an embodiment. Specifically, a portion of a microarray 302 is shown with several wells 304 conformally coated with the multi-layered nanostructure coating. As shown in cross-section A-A′, the multi-layered nanostructure coating is conformally applied to a bottom 306 and side walls 308 of the wells 304 in the PCR microarray 302. Although FIG. 3 illustrates a coating on both the bottom 306 and the side walls 308 of the wells, embodiments are not limited thereto, and the coating may fully or partially cover either the bottom or the side walls, or both the bottom and the side walls of the wells.

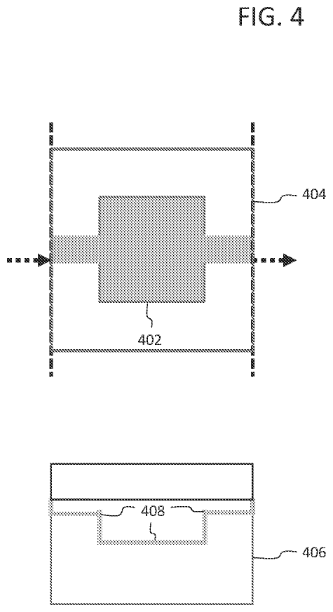

FIG. 4 is a diagram illustrating nanostructure integration to a lab-on-a-chip platform, according to an embodiment. Specifically, the multi-layered nanostructure coating is conformally applied to a sealed PCR reaction chamber 402 in a section of a PCR microfluidic chip 404. The PCR microfluidic chip 404 includes an extraction section that obtains a bio-sample, an amplification section, and a detection section that provides an output. The PCR reaction chamber 402 is within the amplification section of the PCR microfluidic chip 404. A cross-section 406 of the sealed PCR reaction chamber 402 shows that all bottom and side surfaces 408 of the sealed PCR reaction chamber 402 are conformally coated, however, embodiments are not limited thereto, and the coating may fully or partially cover one or more of the surfaces of the sealed PCR reaction chamber 402.

Dimensions of individual nanostructures within the sub-micrometer multi-layered nanostructure coating may be configured to allow for radiative coupling (i.e., suppressed fluorescence quenching) with fluorescent labels used in NA amplification reactions spanning an entire ultraviolet (UV)-visible (VIS)-near-infrared (NIR) spectrum (300-1000 nm).

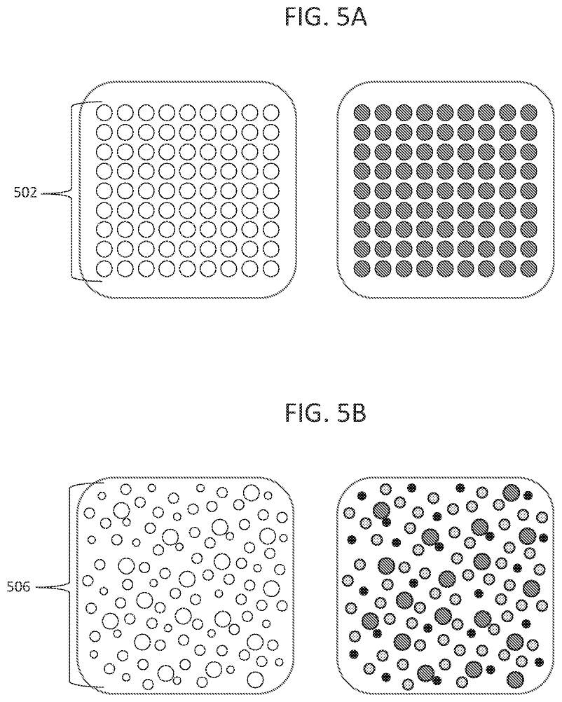

FIGS. 5A and 5B are diagrams illustrating nanostructures for fluorescence enhancement, according to an embodiment. Nanostructures can be designed for specific applications based on the nature of the target(s), type of fluorophore(s), and/or excitation/emission characteristics. FIG. 5A illustrates ordered periodic nanostructures 502 of substantially the same or substantially similar size and dimension that may provide single wavelength enhancement with high sensitivity and a single fluorophore/target (illustrated as a single shade in FIG. 5A). FIG. 5B illustrates disordered random nanostructures 504 of multiple sizes, heights, and spacings that may provide broadband enhancement (e.g., enhancement for multiple wavelengths) with lower sensitivity and multiple fluorophores/targets (illustrated as multiple shades in FIG. 5B). Although a specific configuration of shapes are provided herein, the disclosure is not limited thereto, and embodiments may include a variety of sizes, heights, and spacings. The shape of each sub-micrometer nanostructure may be circular, spherical, ellipsoidal, square, rectangular, tapered, or any combination thereof.

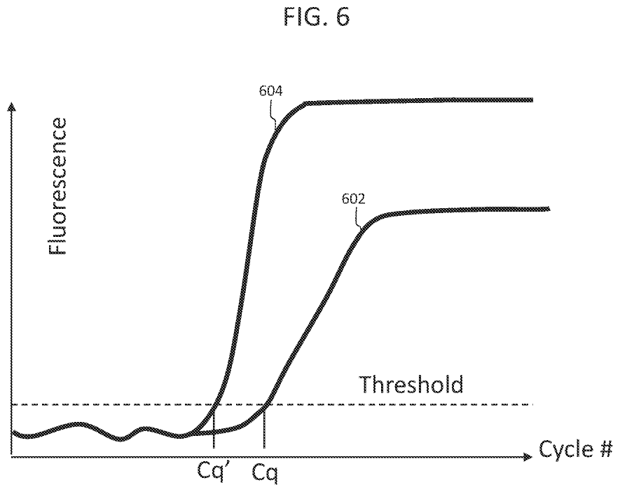

FIG. 6 is a diagram illustrating a comparison of fluorescence versus a number of cycles during NA amplification with and without nanostructures, according to an embodiment. A first fluorescence signal 602 is produced from an amplification reaction chamber without a sub-micrometer multi-layered nanostructure coating. A first minimum number of cycles C_{q} of creating copies of a target NA is required for the first fluorescence signal 602 to overcome the detection threshold. A second fluorescence signal 604 is produced from an amplification reaction chamber with a sub-micrometer multi-layered nanostructure coating. A second minimum number of cycles C_{q}′ of creating copies of a target NA is required for the second fluorescence signal 604 to overcome the detection threshold. Accordingly, in real-time assays, plasmon-enhanced fluorescence from the nanostructures can result in enhancement of the fluorescence from the NA amplification assay leading to a shorter number of cycles required to reach the threshold (i.e., lower critical cycle C_{q}′ versus C_{q}). This results in faster conclusive detection of the fluorescent signal, and thus, the target sequence in the amplified NA.

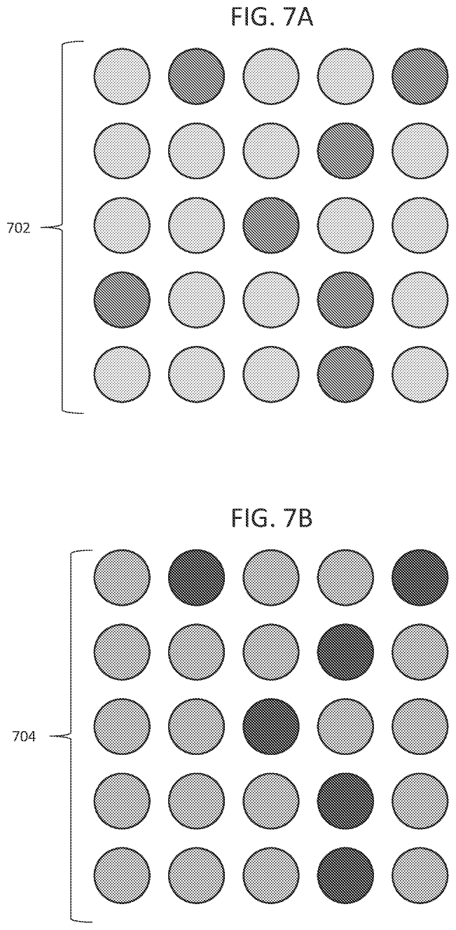

FIGS. 7A and 7B are diagrams illustrating an end-point digital assay comparison of fluorescence with and without nanostructures, according to an embodiment. Plasmon-enhanced fluorescence from the nanostructures can result in enhancement of the fluorescence from the NA amplification assay leading to improved signal contrast during end-point measurements. Specifically, FIG. 7A illustrates end-point assay fluorescence resulting from NA amplification in reaction chambers 702 without a sub-micrometer multi-layered nanostructure coating, and FIG. 7B illustrates an enhanced end-point assay fluorescence resulting from NA amplification in reaction chambers 704 with a sub-micrometer multi-layered nanostructure coating.

Nanostructures may be made of plasmonic materials capable of configurable plasmon-enhanced fluorescence spanning the entire UV-VIS-NIR spectrum (300-1000 nm) for excitation from any UV-VIS-NIR light source(s) including, but not limited to, light-emitting diodes (LEDs), lasers, and all broadband light source(s).

The system may be used for all forms of fluorescence-based quantitative measurements using reporters such as SYBR green dyes and TaqMan probes, as well as all other chemistries relying on fluorescence.

Nanostructures may also be made of plasmonic materials capable of configurable plasmon-enhanced fluorescence to couple with fluorescent dyes including, but not limited to, 6-Carobxyfluorescein (6-FAM) (excitation/emission (ex/em) 495 nm/520 nm), JOE (ex/em 529 nm/555 nm), tetrachlorofluorescein (TET) (ex/em 521 nm/536 nm), VIC (ex/em 538 nm/554 nm), hexachlorofluorescein (HEX) (ex/em 535 nm/556 nm), cyanine (Cy) 3 (ex/em 550 nm/570 nm), Cy 3.5 (ex/em 581 nm/594 nm), Texas Red (ex/em 597 nm/616 nm), Cy 5 (ex/em 650 nm/670 nm), and Cy 5.5 (ex/em 675 nm/694 nm).

The sub-micrometer nanostructure may include metal or doped semiconductor selected from a group including aluminum (Al), gold (Au), silver (Ag), titanium (Ti), tungsten (W), copper (Cu), palladium (Pd), tantalum (Ta), tantalum nitride (TaN), titanium nitride (TiN), Niobium (Nb), p-doped silicon (p Si), or any combination thereof.

Within the sub-micrometer multi-layered nanostructured coating, films made of Al may be used due to their well-understood plasmonic behavior. Typically, Al is less expensive than noble metals and is compatible with complementary metal oxide semiconductor (CMOS) processes. Further, the use of metal-insulator-metal (MIM) architectures may be beneficial for fluorescence enhancement. Thus, an Al film leads to considerable enhancement of fluorescence.

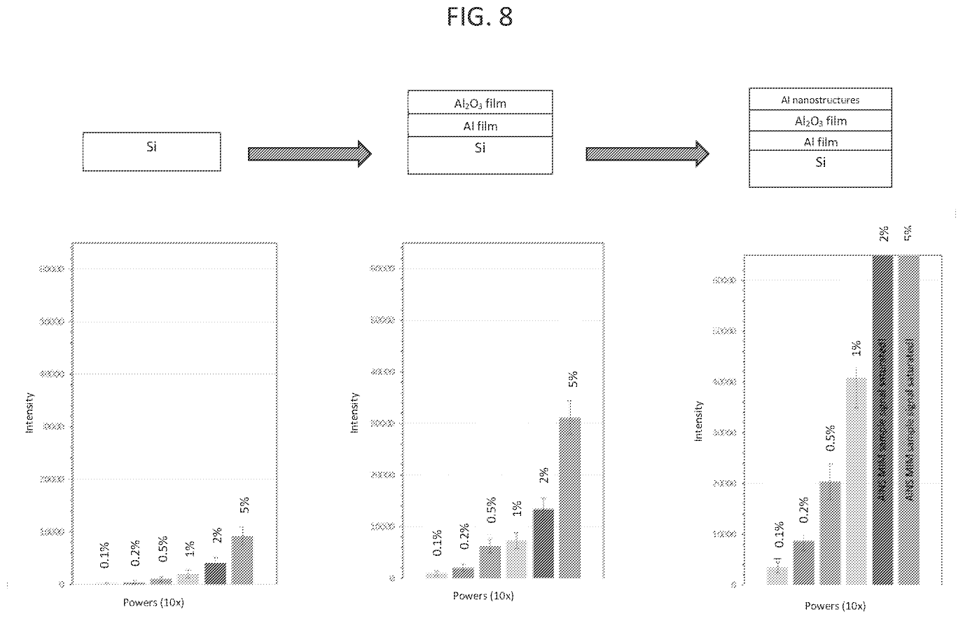

FIG. 8 is a diagram illustrating film optimization for coating an amplification reaction chamber, according to an embodiment. Specifically, a fluorescence intensity is shown for different powers of an excitation light source applied to an amplification reaction chamber, for each of a plain Si surface, an Si surface with a metal film and a dielectric film, and a sub-micrometer multi-layered nanostructure film. Although an Si surface is described with reference to FIG. 8, alternative embodiments may include a surface consisting of plastic materials such as polypropylene of which PCR reaction containers are made.

When a metal film (e.g., approximately 20 nm Al) and a dielectric film (e.g., approximately 15 nm aluminum oxide (Al₂O₃)) are layered on the Si surface, a fluorescence intensity enhancement over Si alone is shown for each of the different excitation light source powers. For example, an approximate power of 0.1% shows a fluorescence intensity enhancement of approximately 6.5 fold, an approximate power of 0.2% shows a fluorescence intensity enhancement of approximately 5.8 fold, an approximate power of 0.5% shows a fluorescence intensity enhancement of approximately 6.3 fold, an approximate power of 1% shows a fluorescence intensity enhancement of approximately 3.7 fold, an approximate power of 2% shows a fluorescence intensity enhancement of approximately 3.3 fold, and an approximate power of 5% shows a fluorescence intensity enhancement of approximately 3.4 fold.

When a nanostructure film (e.g., approximately 20 nm Al nanostructure) is layered over the Al₂O₃ film, intensity enhancement over Si alone is shown for each of the different excitation light source powers. For example, an approximate power of 0.1% shows a fluorescence intensity enhancement of approximately 25 fold, an approximate power of 0.2% shows a fluorescence intensity enhancement of approximately 25 fold, an approximate power of 0.5% shows a fluorescence intensity enhancement of approximately 20 fold, an approximate power of 1% shows a fluorescence intensity enhancement of approximately 20 fold, and an approximate power of 2% and an approximate power of 5% shows that the fluorescence signal is fully saturated.

Although approximate film thicknesses and powers are described with reference to FIG. 8, these are exemplary only and do not limit the disclosure. Specifically, embodiments are not limited to the approximate thicknesses and powers described herein, and thicknesses and powers may vary while still providing described benefits of the disclosure.

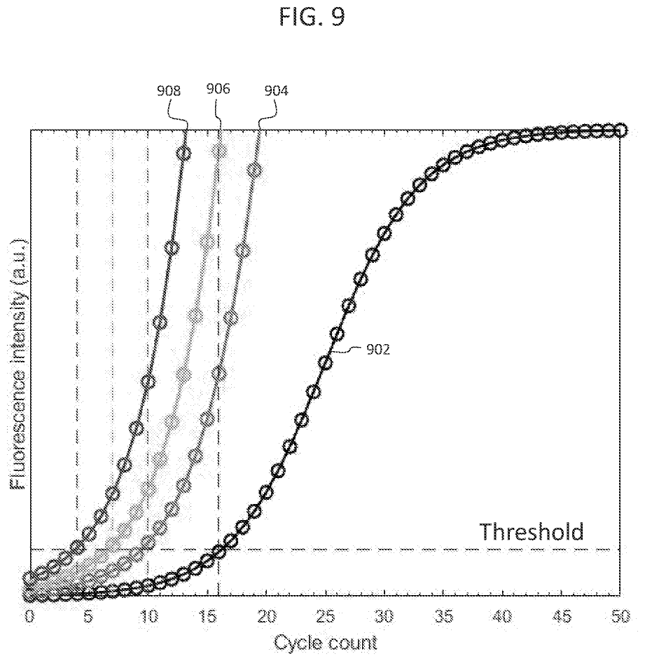

FIG. 9 is a diagram illustrating a comparison of fluorescence versus a number of cycles during NA amplification at different fluorescence enhancements, according to an embodiment. A first fluorescence signal 902 is produced from a reaction chamber without a sub-micrometer multi-layered nanostructure coating, and no resulting fluorescence enhancement. The first fluorescence signal 902 reaches a fluorescence detection threshold at 16 cycles. A second fluorescence signal 904 is produced from a reaction chamber with a sub-micrometer multi-layered nanostructure coating and a fluorescence enhancement increased by approximately 5×. The second fluorescence signal 904 reaches the fluorescence detection threshold at 10 cycles. A third fluorescence signal 906 is produced from a reaction chamber with a sub-micrometer multi-layered nanostructure coating and a fluorescence enhancement increased by approximately 10×. The third fluorescence signal 906 reaches the fluorescence detection threshold at 7 cycles. A fourth fluorescence signal 908 is produced from a reaction chamber with a sub-micrometer multi-layered nanostructure coating and a fluorescence enhancement increased by approximately 20×. The fourth fluorescence signal 908 reaches the fluorescence detection threshold at 4 cycles.

Although approximate fluorescence enhancements are described with reference to FIG. 9, these are exemplary only. Embodiments are not limited to the approximate fluorescence enhancements described herein, and they may vary while still providing described benefits of the disclosure

Nanostructure plasmonic enhancement results in early readout, lower critical cycle (C_{q}), and faster assay time. Nanostructures can be optimized for narrowband and broadband fluorescence enhancement towards a single fluorophore/target and multiple fluorophores/targets. Nanostructure geometric parameters can be configured through traditional integrated circuit (IC) fabrication processes to impact photothermal heating, plasmonic enhancement, and surface hydrophilicity characteristics.

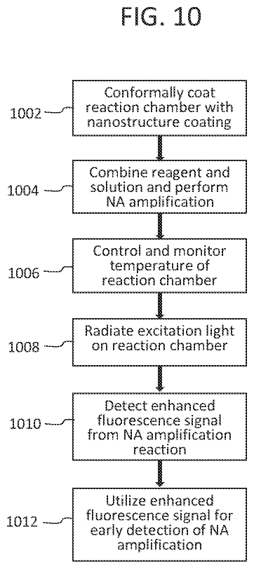

FIG. 10 is a flowchart illustrating a method for performing NA amplification in gene analysis, according to an embodiment. At 1002, an amplification reaction chamber is conformally coated with a multi-layered nanostructure coating having sub-micrometer nanostructures. The multi-layer nanostructure coating may include an Si base, a metal layer disposed on the Si base, a dielectric layer disposed on the metal layer, and a nanostructure layer disposed on the dielectric layer and including the sub-micrometer nanostructures. The metal layer may be composed of Al, and the dielectric layer may be composed of Al₂O₃.

At 1004, an amplification reagent and solution including at least a bio-sample are combined to form an amplification reaction mixture and perform NA amplification in the amplification reaction chamber. At 1006, a temperature of the amplification reaction chamber is controlled and monitored during an NA amplification reaction. At 1008, an excitation light is radiated on the amplification reaction chamber to allow for radiative coupling with fluorescent labels in the NA amplification reaction and generation of a fluorescence signal.

At 1010, the fluorescence signal from the NA amplification reaction is detected from the amplification reaction chamber. The fluorescence signal is enhanced based on the plasmonic material and/or the geometric dimensions of the sub-micrometer nanostructures. The sub-micrometer nanostructures have substantially similar geometric dimensions for narrowband fluorescence enhancement, or various geometric dimensions for broadband fluorescence enhancement. The plasmonic material may be a metal or a doped semiconductor. At 1012, the enhanced fluorescence signal is utilized for early detection of NA amplification.

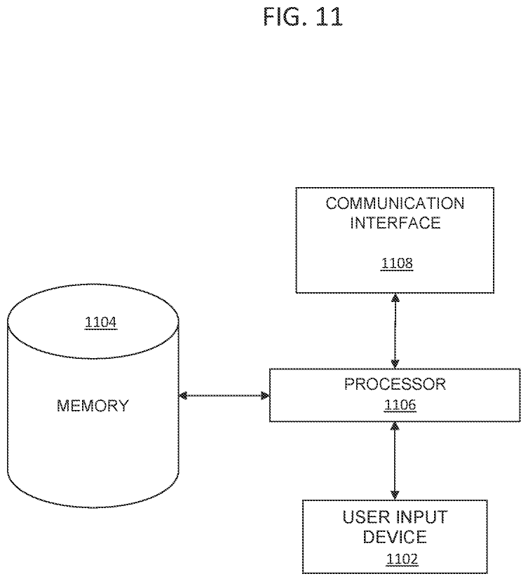

FIG. 11 is a block diagram illustrating a controller for controlling a gene analysis system, according to an embodiment. The processor or controller may include at least one user input device 1102 and a memory 1104. The memory 1104 may include instructions that allow a processor 1106 to control IC fabrication of nanostructures, control coating of an amplification reaction chamber with a multi-layered nanostructure coating, control a temperature control device to control a temperature of the amplification reaction chamber during an NA amplification reaction, control a monitoring device to monitor the temperature of the amplification reaction chamber, control an illumination device to radiate an excitation light on the amplification reaction chamber, and/or control a detection device to detect a fluorescence signal from the NA amplification reaction.

Although certain embodiments of the present disclosure have been described in the detailed description of the present disclosure, the present disclosure may be modified in various forms without departing from the scope of the present disclosure. Thus, the scope of the present disclosure shall not be determined merely based on the described embodiments, but rather determined based on the accompanying claims and equivalents thereto.

What is claimed is:

1. A multi-layered nanostructure coating comprising:
a metal layer;
a dielectric layer disposed on the metal layer; and
a nanostructure layer disposed on the dielectric layer, the nanostructure layer comprising sub-micrometer nanostructures that enhance fluorescence in a nucleic acid (NA) amplification reaction based on at least one of plasmonic material of which the sub-micrometer nanostructures are made and geometric dimensions of the sub-micrometer nanostructures.

2. The multi-layered nanostructure coating of claim 1, further comprising a base on which the metal layer is disposed.

3. The multi-layered nanostructure coating of claim 1, wherein the geometric dimensions allow for radiative coupling with fluorescent labels used in the NA amplification reaction.

4. The multi-layered nanostructure coating of claim 3, wherein each sub-micrometer nanostructure has substantially similar geometric dimensions for narrowband fluorescence enhancement.

5. The multi-layered nanostructure coating of claim 3, wherein the sub-micrometer nanostructures have various geometric dimensions for broadband fluorescence enhancement.

6. The multi-layered nanostructure coating of claim 1, wherein the metal layer is composed of aluminum, and the dielectric layer is composed of aluminum oxide.

7. The multi-layered nanostructure coating of claim 1, wherein the plasmonic material comprises a metal or a doped semiconductor.

8. An amplification reaction chamber of a gene analysis system, the amplification reaction chamber comprising:
an interior surface within which a nucleic acid (NA) amplification reaction is performed; and
a multi-layered nanostructure coating conformally applied to at least a portion of the interior surface and comprising sub-micrometer nanostructures that enhance fluorescence in the NA amplification reaction based on at least one of plasmonic material of which the sub-micrometer nanostructures are made and geometric dimensions of the sub-micrometer nanostructures.

9. The amplification reaction chamber of claim 8, wherein the multi-layered nanostructure coating comprises:
a base;
a metal layer disposed on the base;
a dielectric layer disposed on the metal layer; and
a nanostructure layer disposed on the dielectric layer and comprising the sub-micrometer nanostructures.

10. The amplification reaction chamber of claim 9, wherein the metal layer is composed of aluminum, and the dielectric layer is composed of aluminum oxide.

11. The amplification reaction chamber of claim 8, wherein the geometric dimensions allow for radiative coupling with fluorescent labels used in the NA amplification reaction.

12. The amplification reaction chamber of claim 11, wherein the sub-micrometer nanostructures have substantially similar geometric dimensions for narrowband fluorescence enhancement.

13. The amplification reaction chamber of claim 11, wherein the sub-micrometer nanostructures have various geometric dimensions for broadband fluorescence enhancement.

14. The amplification reaction chamber of claim 8, wherein the plasmonic material comprises a metal or a doped semiconductor.

15. A method for nucleic acid (NA) amplification in a gene analysis system, the method comprising:
conformally coating an amplification reaction chamber with a multi-layered nanostructure coating comprising sub-micrometer nanostructures;
combining an amplification reagent and a solution including at least a bio-sample in the amplification reaction chamber to perform NA amplification; and
detecting, from the amplification reaction chamber, a fluorescence signal from an NA amplification reaction, wherein the fluorescence signal is enhanced based on at least one of plasmonic material of which the sub-micrometer nanostructures are made and geometric dimensions of the sub-micrometer nanostructures.

16. The method of claim 15, further comprising:
controlling and monitoring a temperature of the amplification reaction chamber during the NA amplification reaction; and
radiating an excitation light on the amplification reaction chamber to allow for radiative coupling with fluorescent labels in the NA amplification reaction and generation of the fluorescence signal.

17. The method of claim 15, wherein the multi-layer nanostructure coating comprises:
a base;
a metal layer disposed on the base;
a dielectric layer disposed on the metal layer; and
a nanostructure layer disposed on the dielectric layer and comprising the sub-micrometer nanostructures.

18. The method of claim 17, wherein the metal layer is composed of aluminum, and the dielectric layer is composed of aluminum oxide.

19. The method of claim 15, wherein:
the sub-micrometer nanostructures have substantially similar geometric dimensions for narrowband fluorescence enhancement; or
the sub-micrometer nanostructures have various geometric dimensions for broadband fluorescence enhancement.

20. The method of claim 15, wherein the plasmonic material comprises a metal or a doped semiconductor.
